SEQUENCE LISTING (2 sequences)

SEQ ID NO: 1 - protein, 385 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLGEDPYYTENGGGQGYSSGPGTSPEAQGKASVNRGAQENGTGQATSRNGHSARQHVVADTEL

SEQ ID NO: 2 - protein, 1304 aa, Homo sapiens
MYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

(19) United States
(12) Patent Application Publication
(10) Pub. No.: US 2012/0177618 A1
(43) Pub. Date: Jul. 12, 2012

(54) HEMATOPOIETIC STROMAL PROGENITOR CELLS AND USES THEREOF

(75) Inventors: Biju Parekkadan, Cambridge, MA (US); John Miles Milwid, Cambridge, MA (US); Martin Leon Yarmush, Newton, MA (US)

(73) Assignees: Massachusetts Institute of Technology, Ca,brodge, MA (US); The General Hospital Corporation d/b/a Massachusetts General Hospital, Boston, MA (US)

(21) Appl. No.: 13/383,608

(22) PCT Filed: Jul. 14, 2010
(86) PCT No.: PCT/US10/01978

§ 371 Date: Mar. 26, 2012

Related U.S. Application Data
(60) Provisional application No. 61/225,272, filed on Jul. 14, 2009.

(51) Int. Cl. A61K 35/14; C12N 5/0775; A61P 19/00; C12N 5/0789

(52) U.S. Cl. 424/93.7; 435/372

(57) ABSTRACT

The invention provides compositions and methods for modulating disease using hematopoietic stromal progenitor cells derived from hematopoietic tissues. The invention further provides methods for isolating, storing and culturing hematopoietic stromal progenitor cells.

### RELATED APPLICATIONS

This application claims the benefit and the right to priority of U.S. Provisional Application No. 61/225,272 filed Jul. 14, 2009, entitled MESENCHYMAL STEM CELLS FROM ONE OR MORE HEMATOPOIETIC CELLS, the entire contents of which are incorporated by reference herein.

### GOVERNMENT SUPPORT

This invention was made in part with government support under grant numbers DK059766, DK08942 and 2-T32-HG002295-06A2 from the National Institutes of Health. The Government has certain rights in the invention.

### FIELD OF THE INVENTION

The invention relates to mesenchymal stem cell precursor cells, and methods of their isolation and use.

### BACKGROUND OF THE INVENTION

Mesenchymal cells are a critical component of tissues and serve as supportive elements to organ function by secreting growth factor, remodeling extracellular matrix, and modulating tissue immune responses. Dysfunction of mesenchymal cells has been directly associated with the promotion of hematological diseases such as multiple myeloma (Dierks, Grbic et al. 2007) and myelodysplastic syndromes (Raaijmakers, Mukherjee et al.) and the number and location of mesenchymal cells are considered a prognostic indicator of cancer progression (Hoshida, Villanueva et al. 2008). During tissue injury, fibroblasts participate in wound healing by producing extracellular matrix (ECM) proteins, responding to and synthesizing chemical mediators of inflammation, and contracting the wound bed by differentiating into activated myofibroblasts. Clinically, these mesenchymal cells have been increasingly explored in therapeutic trials for regenerative medicine (Horwitz, Prockop et al. 1999; Au, Tam et al. 2008) and immune-mediated diseases (Parekkadan and Milwid; Aggarwal and Pittenger 2005; Beyth, Borovsky et al. 2005) based on their pleiotropic functions.

The origin of tissue fibroblasts and myofibroblasts has been elusive given the inability to track the fate of these cells in vivo because commonly shared markers with other lineages. A non-hematopoietic class of mesenchymal cells from the bone marrow was previously identified. These cells were assayed by their ability to form fibroblastic colonies in vitro upon plating of whole bone marrow in basal medium cultures. Cells giving rise to such in vitro colonies, referred to as fibroblast colony-forming units (CFU-F), were detected in a number of tissues, including bone marrow, spleen, thymus, lymph node, and peritoneal and pleural fluids. Once expanded in culture these cells are negative for the pan-hematopoietic marker, CD45, and the hematopoietic progenitor cell marker, CD34 (Pittenger, Mackay et al. 1999).

### SUMMARY OF THE INVENTION

The invention is premised in part on the novel and unexpected finding of a class of mesenchymal progenitor cells that express the CD34 marker (i.e., they are CD34+). Mesenchymal progenitor cells, including bone marrow derived mesenchymal stem cells, have previously been characterized as lacking CD34 expression. It has also been found, according to the invention, that the mesenchymal progenitor cells of the invention have superior proliferative and differentiative capacity than earlier isolated mesenchymal progenitor cell populations. These progenitor cells are referred to herein as hematopoietic stromal cell progenitor cells.

The invention provides an isolated population of CD34+ hematopoietic stromal progenitor cells, methods of isolating such cells from, inter alia, hematopoietic tissues such as bone marrow, and methods of use thereof.

In one aspect, the invention provides an isolated cell population comprising enriched CD34+ human hematopoietic stromal progenitor cells. The progenitor cells are enriched in the population 2-fold, 3-fold, 4-fold, 5-fold, 10-fold, or more compared to a starting population from which the progenitor cells derived. An example of the starting population is bone marrow. In preferred embodiments, the cell populations of the invention are human cell populations. In certain embodiments, the progenitor cells are derived from human bone marrow. In certain embodiments, the progenitor cells are derived from other hematopoietic tissues such as peripheral blood, umbilical cord blood, placental tissue, fetal liver tissue, yolk sac, and the like.

In another aspect, the invention provides an isolated cell population comprising enriched CD34+ progenitor cells capable of differentiation into one or more mesenchymal cell lineages. Mesenchymal cell lineages include but are not limited to neuronal lineages, adipocytes, chondrocytes, osteoblasts, myocytes, and cardiac tissue cells. The progenitor cells are enriched in the population at 2-fold, 3-fold, 4-fold, 5-fold, 10-fold, or more compared to a starting population from which the progenitor cells derived. An example of the starting population is bone marrow. In preferred embodiments, the cell populations of the invention are human cell populations. In other embodiments, the cell population of the invention is non-human and may derive from companion animals such as dogs, cats, and the like, or agricultural animals such as horses, cattle, and the like. In certain embodiments, the progenitor cells are derived from human bone marrow, while in other embodiments the cells are derived from other hematopoietic tissue.

In some embodiments, the enriched CD34+ human hematopoietic stromal progenitor cells are adherent cells. In some embodiments, the enriched CD34+ progenitor cells capable of differentiation into one or more mesenchymal cell lineages are adherent cells. As used herein, adherent cells are cells that are adherent to tissue culture dishes or other treated solid or semi-solid supports.

In another aspect, the invention provides an in vitro culture comprising any of the foregoing isolated cell populations or populations enriched for CD34+ progenitor cells. The culture may comprise adherent cells. The culture may further comprise cells from one or more mesenchymal lineages, including but not limited to those recited herein, derived from the CD34+ progenitor cells.

In another aspect, the invention provides a composition comprising an isolated, enriched population of CD34+ human hematopoietic stromal progenitor cells. In another aspect, the invention provides a composition comprising an isolated, enriched population of CD34+ progenitor cells capable of differentiation into one or more mesenchymal cell lineages. In some embodiments, the composition further comprises culture medium, cryopreservant(s), and/or a pharmaceutically acceptable carrier. In important embodiments, the composition is sterile and suitable for in vivo use. In some embodiments, the CD34+ progenitor cells are derived from human bone marrow.

In another aspect, the invention provides a method comprising isolating CD34+ cells from a bone marrow cell population, culturing the isolated CD34+ cells in an in vitro culture, and separating, from the in vitro culture, adherent cells from non-adherent cells.

In one embodiment, the bone marrow cells are human bone marrow cells.

In one embodiment, the method further comprises harvesting the adherent cells from the in vitro culture. In one embodiment, the method further comprises isolating CD29-negative cells from the adherent cells from the in vitro culture. In one embodiment, the method further comprises isolating CD106-negative cells from the adherent cells from the in vitro culture.

In another aspect, the invention provides an isolated cell population prepared by any of the foregoing methods.

In another aspect, the invention provides a method comprising administering to a subject in need of a mesenchymal stem cell transplant a therapeutically effective amount of isolated hematopoietic stromal progenitor cells. In some embodiments, the isolated hematopoietic stromal progenitor cells are CD34+. In some embodiments, the isolated hematopoietic stromal progenitor cells are adherent cells. In some embodiments, the isolated hematopoietic stromal progenitor cells are CD29-negative. In some embodiments, the isolated hematopoietic stromal progenitor cells are CD106-negative. In some embodiments, the isolated hematopoietic stromal progenitor cells are adherent cells that are CD29-negative and CD106-negative.

In some embodiments, the subject is not in need of a hematopoietic stem cell transplant or hematopoietic reconstitution. A subject not in need of a hematopoietic stem cell transplant or hematopoietic reconstitution, as used herein, may be a subject having normal blood cell numbers, a subject that is not immunocompromised, or a subject who has not been exposed to radiation, chemotherapy or other physical or chemical agent that reduces blood cell numbers.

In some embodiments, the subject in need of a mesenchymal stem cell transplant is a subject having a condition characterized by a mesenchymal cell lineage defect. An example is a connective tissue defect. Examples of connective tissue defects include bone defects, cartilage defects, and the like. In some embodiments, the subject is experiencing an aberrant immune response but is not in need of a hematopoietic stem cell transplant or of hematopoietic reconstitution.

In still another aspect, the invention provides a method for culturing isolated hematopoietic stromal progenitor cells comprising isolating hematopoietic stromal progenitor cell based on a hematopoietic surface antigen (e.g., CD34 or CD45) and culturing the cells in a defined environment, including a defined culture surface (e.g., a treated tissue culture surface) and/or a defined culture medium such as DMEM with supplemented nutrients, antibiotics, and serum.

In one embodiment, the hematopoietic surface antigen used to physically separate the hematopoietic stromal progenitor cell from a population is CD34. In another embodiment, the hematopoietic surface antigen used to physically separate the hematopoietic stromal progenitor cell from a population is CD45. In one embodiment, the isolated hematopoietic stromal progenitor cell is derived from human bone marrow.

In another aspect, the invention provides a method for treating a subject having or at risk of developing an orthopedic disease comprising administering to a subject in need thereof an isolated hematopoietic stromal progenitor cell or cell population in an effective amount to treat the subject. The cell population may be prepared according to any of the foregoing methods, but it is not so limited.

In another aspect, the invention provides a method for modulating an immune response comprising administering to a subject in need thereof an isolated hematopoietic stromal progenitor cell or cell population in an effective amount to treat the subject. The cell population may be prepared according to any of the foregoing methods, but it is not so limited.

In another aspect, the invention provides a method for diagnosing a disease comprising isolating and enumerating hematopoietic stromal progenitor cells in a sample from a subject, wherein a below normal number of hematopoietic stromal progenitor cells in a sample from the subject indicates that the subject is in need of the isolated hematopoietic stromal progenitor cells of the invention. The method may further comprise administering to said subject a population of isolated hematopoietic stromal progenitor cells of the invention.

These and other embodiments of the invention will be described in greater detail herein:

### BRIEF DESCRIPTION OF THE DRAWINGS

The drawings provided herein are not intended to be drawn to scale. In the drawings, each identical or nearly identical component that is illustrated in various figures is represented by a like numeral. For purposes of clarity, not every component may be labeled in every drawing.

FIG. 1. Colony forming unit-fibroblast assay for different seeding densities of CD34+, CD45+ and whole bone marrow fractions.

FIG. 2. Morphology of hematopoietic stromal progenitor cells.

FIG. 3. Immunophenotype of hematopoietic stromal progenitor cells.

FIG. 4. Osteogenic/adipogenic potential of the adherent cells in culture from the three fractions.

FIG. 5. Hematopoietic stromal progenitor cells derived from CD34⁺ and CD45⁺ populations posses the same capacity for ectopic marrow formation as whole bone marrow mesenchymal cells. HA/TCP mixture with stromal cells from either three fractions were subcutaneously implanted into immunocompromised mice. Human skin dermal fibroblasts were used as control. Representative images of implants after 8 weeks. N=3 per group Bar graph shows quantitative image analysis of the percentage of nucleated cells per field area.

### DETAILED DESCRIPTION OF THE INVENTION

The invention is based in part on the discovery of a stromal progenitor cells derived from hematopoietic tissue having a novel and unexpected phenotype and functional characteristics. These novel, previously undisclosed progenitor cells are referred to herein as hematopoietic stromal progenitor cells. Although capable of differentiation into mesenchymal lineages, these progenitor cells express one or both hematopoietic markers CD34 or CD45, and can be isolated from the bone marrow.

Various aspects and embodiments of the invention relate to the isolation, analysis, manipulation, prophylactic or therapeutic use, and/or screening of hematopoietic stromal progenitor cells. As defined herein, a hematopoietic stromal progenitor cell is a CD34+ cell having the capacity to differentiate into neuronal cells, adipocytes, chondrocytes, osteoblasts, myocytes, cardiac tissue, and other endothelial and/or epithelial cells. In some embodiments, the progenitor cells are able to differentiate into two or more, three or more, four or more, five or more, or six or more such lineages. The hematopoietic stromal progenitor cell may be obtained (or harvested) from hematopoietic tissue such as bone marrow, although the invention is not so limited.

As described herein, the invention further provides isolated cell populations comprising daughter cells of CD34+ hematopoietic stromal progenitor cells. Such daughter cells may or may not express CD34 on their cell surface. In some embodiments, such daughter cells are adherent cells (e.g., they adhere in culture to a treated tissue culture surface). In some embodiments, such daughter cells are CD29-negative and/or CD106-negative, compared to unfractionated bone marrow nucleated cells. It is to be understood that for the sake of convenience the invention is described in the context of hematopoietic stromal progenitor cells, however, it is intended that the various aspects and embodiments set forth herein including the compositions, cultures and methods of the invention apply equally to isolated populations of daughter cells of CD34+ hematopoietic stromal progenitor cells.

The hematopoietic stromal progenitor cell may also be characterized as being an adherent cell type. Accordingly, such cells may be obtained from, for example, bone marrow by isolating CD34+ cells from the bone marrow cell population, and optionally contacting such CD34+ cells to adherent surfaces such as but not limited to tissue culture surfaces. Additionally or alternatively, the CD34+ cells may be separated based on cell surface expression of one or more other markers such as CD29 and CD 106. Some embodiments of the invention therefore relate to cells that are CD34+ and CD29− (i.e., positive for CD34 and negative for CD29). Some embodiments of the invention relate to cells that are CD34+ and CD106− (i.e., positive for CD34 and negative for CD106). Still other embodiments relate to cells that are CD34+, CD29− and CD106− (i.e., positive for CD34, and negative for CD29 and CD 106).

The expression of CD34 and/or the lack of expression of CD29 and CD106 distinguish the hematopoietic stromal progenitor cells of the invention from previously described populations of mesenchymal precursors. As an example, previously described mesenchymal precursor populations have been characterized as CD34− (negative for CD34), CD45− (negative for CD45), CD29+ (positive for CD29) and CD106+ (positive for CD106).

The invention provides in yet another aspect a mesenchymal progenitor population that is CD45+ (i.e., positive for CD45).

The invention further provides an isolated cell population that is produced by culturing CD34+ bone marrow cells in vitro in order to generate an adherent cell population, and harvesting the adherent population. The method may further comprise isolating cells that are negative for CD29 and/or CD106 from the adherent cell population. In this manner, the invention provides a method for producing an isolated population of hematopoietic stromal progenitor cells that are enriched, relative to a starting population such as bone marrow, by 2-fold, 3-fold, 4-fold, 5-fold, 10-fold, 20-fold, 50-fold, 100-fold, 200-fold, 500-fold, or more. Mesenchymal precursor cells have previously been characterized as CD29+ and CD106+ and thus the characterization of progenitor cells that are negative for one or both CD29 and CD106 and that can differentiate into mesenchymal cell lineages, as provided by the invention, is novel and unexpected.

Although not intending to be bound by any particular theory or mechanism, it is contemplated that the progenitor cells of the invention are derived from a source that is separate and distinct from that which certain previously characterized mesenchymal progenitor cells have been derived. For example, certain previously described mesenchymal stem cells have been thought to be of mesenchymal origin, while the progenitor cells of the invention may be of hematopoietic origin due to their expression of the hematopoietic cell surface marker CD34 (and optionally CD45). In addition, it is also contemplated that the progenitor cells of the invention may be precursors (and thus give rise) to other previously described mesenchymal progenitor cells. Accordingly, while not being bound by any theory or mechanism, it is contemplated that the progenitor cells of the invention may differentiate into more mature progenitors, and in the process may change their surface phenotype, including for example decreasing expression of CD34 and/or increasing expression of CD29 and/or CD106.

In some embodiments, the hematopoietic stromal progenitor cells may also express other classical hematopoietic markers such as c-kit, Sca-1, CD39, CD59, CD135, CD133, CD38 and/or CDw150.

The progenitors of the invention may further differentiate giving rise to more classically characterized mesenchymal progenitors cells, as described by Wang, Stem Cells 2004; 22 (7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; July 10; 51 (3); 223-33; Yen; Stem Cells; 2005; 23 (1) 3-9.) According to these reports, certain mesenchymal precursors have been characterized as expressing (or being positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-Ll. These mesenchymal precursors have also been characterized as not expressing (and thus being negative for) CD3, CD5, CD6, CD9, CD 10, CD 11 a, CD 14, CD15, CD18, CD21, CD25, CD31, CD34, CD36, CD38, CD45, CD49d, CD50, CD62E, L, S, CD80, CD86, CD95, CD117, CD133, SSEA-1, and ABO.

The hematopoietic stromal progenitor cells of the invention (and their CD29-negative and CD106-negative daughter cells) are distinct from these previously characterized mesenchymal precursors in one or more ways including their expression of CD34, their reduced (or lack of) expression of CD29 and CD106 following in vitro culture, their more rapid cell cycles, higher efficiency in responding to conditions that induce differentiation into bone and fat tissues, and greater anti-inflammatory activity. The CD34+ hematopoietic stromal progenitor cells demonstrate a faster doubling rate in culture as compared to CD34− mesenchymal stem cells. As an example, the CD34+ progenitor cells demonstrated a doubling rate of about 24-26 hours compared to a CD34− progenitor cells which demonstrated a doubling rate of about 48-72 hours. Additionally, CD34+ progenitor cells are capable of extended passage in culture compared to CD34− progenitor cells. As an example, the CD34+ progenitor cells could be passaged for at least 15 passages without senescence, while the CD34− progenitor cells appeared to senesce at about passage 10 under the same culture conditions.

In a preferred embodiment, the hematopoietic stromal progenitor cells are derived from bone marrow, are adherent and are positive for cell surface expression of CD34 and optionally are CD45 positive as well. These cells may be additionally characterized in some embodiments as CD105+ (SH-2+), CD73+ (SH-3+ and SH-4+), and CD14−.

Hematopoietic stromal progenitor cells may be harvested from a number of sources including but not limited to bone marrow and blood. Methods for harvest of hematopoietic stromal progenitor cells from the bone marrow are described in greater detail in the Examples.

As used herein, it is to be understood that aspects and embodiments of the invention relate to cells as well as cell populations, unless otherwise indicated. Thus, where a cell is recited, it is to be understood that a cell population is also contemplated unless otherwise indicated.

As used herein, an isolated hematopoietic stromal progenitor cell is a hematopoietic stromal progenitor cell, as described herein, that has been physically separated from its natural environment, including physical separation from one or more components of its natural environment. Thus, an isolated cell or cell population embraces a cell or a cell population that has been harvested from its normal in vivo environment and/or that has been manipulated in vitro or ex vivo. As an example, isolated hematopoietic stromal progenitor cells may be hematopoietic stromal progenitor cells that have been physically separated from at least 50%, preferably at least 60%, more preferably at least 70%, and even more preferably a least 80% of the cells in the tissue from which the hematopoietic stromal progenitor cells are harvested (e.g., bone marrow).

In some instances, the isolated hematopoietic stromal progenitor cells are present in a population that is at least 85%, at least 90%, at least 95%, at least 96%, at least 97%, at least 98%, at least 99%, or 100% hematopoietic stromal progenitor cells as phenotypically and/or functionally defined herein. Preferably the ratio of hematopoietic stromal progenitor cells to other cells is increased following “isolation”, thereby representing enrichment of the progenitor cells compared to the starting population of cells (e.g., unfractionated bone marrow cells). The degree of enrichment may vary depending upon the isolation strategy. Depending on the isolation method(s) used, the progenitors of the invention may be enriched 2-fold, 3-fold, 4-fold, 5-fold, 6-fold, 7-fold, 8-fold, 9-fold, 10-fold, or more.

Hematopoietic stromal progenitor cells can be isolated using methods known in the art, e.g., from bone marrow mononuclear cells, based on their cell surface expression of CD34, optionally together with their ability adhere to surfaces such as but not limited to tissue culture treated surfaces. The hematopoietic tissue source, such as bone marrow, may be freshly harvested (e.g., from a patient) or it may be obtained from a commercial source.

Isolation of hematopoietic stromal progenitor cells based on cell surface expression of CD34 may be performed by any number of methods routinely used in the art. Typically, these methods employ antibodies (or antibody fragments) that bind, preferably specifically, to CD34. The antibodies may be conjugated to a detectable label or they may be used together with a secondary antibody, as is routine in the art. The antibodies may be used in a soluble form and allowed to contact and bind to cells that express CD34, followed by contact with a secondary antibody. Fluorescent activated cell sorting (FACS) may then be used to separate CD34+ cells from the remainder of the cell population. Alternatively, the antibodies may be immobilized onto solid surfaces such as plates, wells, beads, columns, and the like. Cell populations are then contacted with and allowed to attach to such solid surfaces via their specificity to the anti-CD34 antibody or antibody fragment. Antibodies and antibody fragments specific for CD34 are commercially available from sources such as R&D Systems, Santa Cruz Biotechnology. Examples include but are not limited to QBend10, 563, HPCA-2, 581, AC136, and Birma K3.

Similar cell separation strategies can be used to further separate the CD34+ cell population prior to or following in vitro culture according to other markers.

One of ordinary skill in the art will readily envision negative selection methods that allow for the isolation of cells that do not express markers such as CD29 and CD106. Briefly, these negative selection methods comprise contacting a cell'population with, for example, an antibody specific for a particular cell surface marker and separating the cells that express the marker from those that do not express the marker. Typically, cells that express the marker will be labeled with a detectable label (and thus separable from those that are not labeled with the detectable label), or they will be bound to a solid support (and thus separable from those that are free is solution).

Commercially available media may be used for the growth, culture and maintenance of hematopoietic stromal progenitor cells. Such media include but are not limited to Dulbecco's modified Eagle's medium (DMEM). Components in such media that are useful for the growth, culture and maintenance of hematopoietic stromal progenitor cells include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine).

Commercially available tissue culture substrates media may be used for the growth, culture and maintenance of hematopoietic stromal progenitor cells. Such substrates include but are not limited to polystyrene. Other such substrates include different extracellular matrix coating such as type I collagen, fibronectin, laminin, Matrigel, decorin, cadherins and other natural products and derivatives of these such as RGD polypeptide motifs. Synthetic materials and plastics can also be used as substrates and include poly-L-lysine, PLGA, PLL, alginate, PMMA and other polymer compositions. Substrates can also be created in 3-dimensions such as polystyrene beads to be used for suspension culture of adherent hematopoietic stromal progenitor cells.

As one example, hematopoietic stromal progenitor cells may be prepared as follows. Bone marrow cells can be fractionated based on the surface marker CD34, and then cultured using Dulbecco's modified Eagle's medium supplemented with 10% fetal calf serum, 100 U/ml penicillin, 100 μg/ml streptomycin, 0.1 mM non-essential amino acids and 1 ng/ml of basic fibroblast growth factor (Life Technologies, Rockville, Md.). After 4 days of culture, non-adherent cells can be removed by washing with PBS. Monolayers of adherent cells are then cultured with medium changes 2-3 times per week. Cells can be passaged using 0.25% trypsin/0.1% EDTA and subcultured at a density of 5×10₃ cells/cm². Hematopoietic stromal progenitor cells can be maintained using methods known in the art (see, e.g., Pittenger et al., Science, 284:143-147 (1999)). A similar process may be used to isolate CD45+ cells progenitor cells from bone marrow, in accordance with the invention.

It will further understood that following the in vitro culture the cultured cells may be further fractionated according to CD29 and/or CD106 cell surface expression, in accordance with the invention. Therefore, according to one embodiment of the invention, the isolation process comprises isolation of CD34+ cells, in vitro culture of such cells to obtain the adherent fraction, and further separation of this population into CD29-negative, CD106-negative, or CD29-negative and CD106-negative populations. Accordingly, one cell population provided by the invention is CD34+, CD29−, CD 106− and adherent. The characterization of these progenitors as negative for CD29 and CD106 is based on a comparison of the level of expression of these markers by whole bone marrow populations, as shown in the Examples. The term “CD34”, as used herein, refers to the “cluster of differentiation molecule 34” which is known in the art. It is bound by any of a number of anti-CD34 antibodies including but not limited to QBend10, 563, HPCA-2, 581, AC136, and Birma K3. Additionally, it has been characterized as having an amino acid sequence selected from the group consisting of GenBank ID: 947, NCBI RefSeq ID: NP_{—}001020280.1, and/or NP_{—}001764.1

The term “CD45”, as used herein, refers to the “cluster of differentiation molecule 45” which is known in the art. CD45 has been further characterized as a protein tyrosine phosphatase receptor. It has been characterized as having an amino acid sequence selected to from the group consisting of GenBank ID: 5788, NCBI RefSeq ID: NP_{—}563580.1, NP_{—}563579.1, NP_{—}563578.1, and/or NP_{—}002829.2.

TABLE 1
Representative sequences of CD34 and CD45

| | |
|---|---|
| Protein | Exemplary Sequence |

(SEQ ID NO: 1)

| | |
|---|---|
| CD34 | MLVRRGARAG PRMPRGWTAL CLLSLLPSGF MSLDNNGTAT PELPTQGTFS |
| | NVSTNVSYQE TTTPSTLGST SLHPVSQHGN EATTNITETT VKFTSTSVIT |
| | SVYGNTNSSV QSQTSVISTV FTTPANVSTP ETTLKPSLSP GNVSDLSTTS |
| | TSLATSPTKP YTSSSPILSD IKAEIKCSGI REVKLTQGIC LEQNKTSSCA |
| | EFKKDRGEGL ARVLCGEEQA DADAGAQVCS LLLAQSEVRP QCLLLVLANR |
| | TEISSKLQLM KKHQSDLKKL GILDFTEQDV ASHQSYSQKT LIALVTSGAL |
| | LAVLGITGYF LMNRRSWSPT GERLGEDPYY TENGGGQGYS SGPGTSPEAQ |
| | GKASVNRGAQ ENGTGQATSR NGHSARQHVV ADTEL |

(SEQ ID NO: 2)

| | |
|---|---|
| CD45 | mylwlkllaf gfafldtevf vtgqsptpsp tglttakmps vplssdplpt httafspast |
| | ferendfset ttslspdnts tqvspdsldn asafnttgvs svqtphlpth adsqtpsagt |
| | dtqtfsgsaa naklnptpgs naisdvpger stastfptdp vspltttlsl ahhssaalpa |
| | rtsnttitan tsdaylnase tttlspsgsa vistttiatt pskptcdeky anitvdylyn |
| | ketklftakl nvnenvecgn ntctnnevhn ltecknasys ishnsctapd ktlildvppg |
| | vekfqlhdct qvekadttic lkwknietft cdtqnityrf qcgnmifdnk eiklenlepe |
| | heykcdseil ynnhkftnas kiiktdfgsp gepqiifcrs eaahqgvitw nppqrsfhnf |
| | tlcyiketek dclnldknli kydlqnlkpy tkyvlslhay iiakvqrngs aamchfttks |
| | appsqvwnmt vsmtsdnsmh vkcrpprdrn gpheryhlev eagntlvrne shkncdfrvk |
| | dlqystdytf kayfhngdyp gepfilhhst synskaliaf lafliivtsi allvvlykiy |
| | dlhkkrscnl deqqelverd dekqlmnvep ihadillety krkiadegrl flaefqsipr |
| | vfskfpikea rkpfnqnknr yvdilpydyn rvelseingd agsnyinasy idgfkeprky |
| | iaaqgprdet vddfwrmiwe qkatvivmvt rceegnrnkc aeywpsmeeg trafgdvvvk |
| | inqhkrcpdy iiqklnivnk kekatgrevt hiqftswpdh gvpedphlll klrrrvnafs |
| | nffsgpivvh csagvgrtgt yigidamleg leaenkvdvy gyvvklrrqr clmvqveaqy |
| | ilihqalvey nqfgetevnl selhpylhnm kkrdppseps pleaefqrlp syrswrtqhi |
| | gnqeenkskn rnsnvipydy nrvplkhele mskesehdsd essdddsdse epskyinasf |
| | imsywkpevm iaaqgplket igdfwqmifq rkvkvivmlt elkhgdqeic aqywgegkqt |
| | ygdievdlkd tdksstytlr vfelrhskrk dsrtvyqyqy tnwsveqlpa epkelismiq |
| | vvkqklpqkn ssegnkhhks tpllihcrdg sqqtgifcal lnllesaete evvdifqvvk |
| | alrkarpgmv stfeqyqfly dviastypaq ngqvkknnhq edkiefdnev dkvkqdancv |
| | nplgapeklp eakeqaegse ptsgtegpeh svngpaspal nqgs |

The isolation methods of the invention may be carried out using any binding partner that is sufficiently specific for a marker of choice. The nature of the binding partner may vary and it may amino acid (e.g., antibody or antibody fragment), nucleic acid (e.g., aptamer), or chemical (e.g., combinatorial library member) in nature, although it is not so limited. A binding partner that is sufficiently specific for a marker of choice is one that binds to the marker of choice with greater specificity than it binds to another marker. Preferably, the binding partner binds to the marker of choice with an affinity that is 2-fold, 3-fold, 4-fold, 5-fold, 10-fold, 20-fold, 50-fold, 100-fold, 500-fold, or 1000-fold more than its affinity for another marker.

In some embodiments, the isolation methods are carried out using antibodies or antibody fragments. As used herein, the term “antibody” refers to any immunoglobulin, whether natural or wholly or partially synthetically produced. All derivatives thereof which maintain specific binding ability are also included in the term. The term also covers any protein having a binding domain that is homologous or largely homologous to an immunoglobulin binding domain. Such proteins may be derived from natural sources, or partly or wholly synthetically produced. In some embodiments, an antibody is monoclonal. In some embodiments, an antibody is polyclonal. In some embodiments, an antibody is a single chain antibody. Those of ordinary skill in the art will appreciate that antibodies may be provided in any of a variety of forms including, for example, humanized, partially humanized, chimeric, chimeric humanized, etc. An antibody may be a member of any immunoglobulin class, including any of the human classes: IgG, IgM, IgA, IgD, and IgE, and of any immunoglobulin subclass (e.g., IgG1, IgG2, IgG3, or IgG4). Typically, an intact antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. A heavy chain constant region is comprised of three or four domains, CH1, CH2, CH3, and CH4, depending on the isotype. Each light chain is comprised of a light chain variable region (abbreviated herein as VL) and a light chain constant region. A light chain constant region is comprised of one domain, CL. VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. Variable regions of heavy and light chains contain a binding domain that interacts with an antigen. Constant regions of antibodies may mediate binding of antibodies to host tissues or factors, including various cells of the immune system (e.g., effector cells) and the first component (Clq) of the classical complement system. As used herein, the terms “antibody fragment” (i.e., “antigen-binding portion”) or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment typically comprises at least about 50 amino acids, at least about 100 amino acids, at least about 150 amino acids, or at least about 200 amino acids.

The invention further provides progenitor cell lysates, according to other embodiments. Hematopoietic stromal progenitor cell lysates may be prepared by any lysis method known in the art provided that the resulting lysate is not toxic to cells. These methods include chemical and/or mechanical methods such as osmotic shock, ultrasound, and shearing of cells. The lysate may be concentrated, filtered, or manipulated in other ways that do not impact its antigen content.

The invention contemplates genetically engineering hematopoietic stromal progenitor cells. This can be accomplished using methods known in the art. Expression vectors to be introduced into hematopoietic stromal progenitor cells will generally include the pertinent sequence, i.e., nucleotide sequences that encode the gene to be expressed, and transcriptional and translational control sequences such as promoters, enhancers, poly A sequences, termination sequences and the like. In some instances, two or more coding sequences may be included in the vector, preferably with an IRES or functionally equivalent sequence located therebetween. The cells being so transduced or transfected may not naturally express one or more of the proteins encoded by the expression vectors or may not express them at suitable levels.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined to each other when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding an antigen of interest. That heterologous DNA (RNA) is placed under operable control of transcriptional elements to permit the expression of the heterologous DNA in the host cell.

Preferred systems for mRNA expression in mammalian cells are those such as pRc/CMV and pcDNA3.1 (available from Invitrogen, Carlsbad, CA) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against PIA (Int. J. Cancer, 67:303-310, 1996). Recombinant vectors including viruses selected from the group consisting of adenoviruses, adeno-associated viruses, poxviruses including vaccinia viruses and attenuated poxviruses such as ALVAC, NYVAC, Semliki Forest virus, Venezuelan equine encephalitis virus, retroviruses, Sindbis virus, Ty virus-like particle, other alphaviruses, VSV, plasmids (e.g., “naked” DNA), bacteria (e.g., the bacterium Bacille Calmette Guerin, attenuated Salmonella), and the like can be used in such delivery, for example, for use as a vaccine.

The invention further provides a hematopoietic stromal progenitor cell conditioned media (HSPC-CM) composition. An HSPC-CM composition can be prepared by culturing a hematopoietic stromal progenitor cell population. The population may be one that has been passaged or one that has just been isolated and cultured. Preferably, it has been passaged and more preferably it is between passage 2-7. The hematopoietic stromal progenitor cells may be cultured at a density of about 1×10⁵ to 1×10′ cells, e.g., about 1×10⁵ to 1×10⁶ cells, 1×10⁶ to 1×10⁷ cells, 1×10⁶ to 9×10⁶cells, 1×10⁶ to 8×10⁶ cells, 1×10⁶ to 7×10⁶ cells, 1×10⁶ to 6×10⁶ cells, 1×10⁶ to 5×10⁶ cells, 1×10⁶ to 4×10⁶ cells, 1×10⁶ to 3×10⁶ cells, and 1×10⁶ to 2×10⁶ cells.

In some embodiments, an HSPC-CM is prepared as follows: (1) wash 70-80% confluent hematopoietic stromal progenitor cells thoroughly with phosphate buffered saline (PBS); (2) Culture hematopoietic stromal progenitor cells for about 12, 24, 36, or 48 hours, e.g., 24 hours in an appropriate volume of serum free culture medium containing DMEM, or an equivalent thereof, supplemented with 0.05% bovine serum albumin (BSA) in a suitable vessel, e.g., a T175 cm² flask, with each vessel/flask at 80% confluency, equivalent to about 5×10³ 15 cells/cm²; and (3) Collect HSPC culture media from (2).

The collected HSPC-CM can be concentrated, e.g., using methods known in the art, for example, ultrafiltration units with a 3 kD cutoff (AMICON Ultra-PL 3, Millipore, Bedford, Mass., USA). For example, the HSPC-CM can be concentrated at least 2-fold to 10-fold, 10-fold to 20-fold, 20-fold to 30-fold, 30-fold to 49-fold, and above. As one example, an HSPC-CM is concentrated 25-fold. In some embodiments, the HSPC-CM comprises culture medium containing DMEM supplemented with 0.05% bovine serum albumin (BSA). In some embodiments, the HSPC-CM composition does not contain any animal serum or other animal products. In some embodiments, the HSPC-CM composition comprises PBS. Alternatively, the HSPC-CM is provided in lyophilized form. In some embodiments, an HSPC-CM can be fractionated by size or by charge.

In some embodiments, for example, an HSPC-CM can be fractionated into heparin sulfate binding and non heparin binding fractions. For example, in heparin sulfate fractionation experiments, a concentrated HSPC-CM can be passed over a heparin column, or other columns e.g., an ion-exchange, size, reverse-phase or other chromatographic separation methods per vendor's instructions. Flow-through and eluted fractions can then be collected separately. The eluted fractions (i.e., the heparin-binding fraction) can then be collected and optionally concentrated, as described above. In some embodiments, an HSPC-CM composition is at least 50%, 60%, 70%, 80%, 90%, and 100% free of non-heparin binding material.

In some embodiments, HSPCs can condition the blood components of a subject by being in contact with a subject's circulation either directly or separated by a semi-permeable membrane within a device.

The invention further contemplates the use of the isolated hematopoietic stromal progenitor cells, the HSPC conditioned media or blood, and/or the HSPC lysates, alone or in any combination for the prevention or treatment of, inter alia, aberrant immune responses and/or conditions resulting therefrom. Subjects to whom these cellular and/or acellular compositions may be administered include those at risk of developing aberrant immune responses (and the conditions resulting therefrom) based on for example a genetic predisposition, or subjects presently having such immune responses. Typically, any subject in need of a mesenchymal stem cell transplant may be treated using the compositions of the invention.

As used herein, an aberrant immune response is one that is upregulated compared to immune responses in a normal subject population. In some important embodiments, the immune response is directed to a self antigen (i.e., an antigen that is encoded in the genome of the subject being treated). The normal subject population is one that does not possess such anti-self immune reactivity, except as may occur for example in cancer immunosurveillance.

Thus, the methods provided herein aim to reduce, diminish, control or completely eliminate such aberrant immune responses. Subjects in need of such immunomodulation include those having or those at risk of developing autoimmune diseases, those having or at risk of graft-versus-host disease, and the like.

Examples of autoimmune diseases include but are not limited to multiple sclerosis, inflammatory bowel disease including Crohn's Disease and ulcerative colitis, rheumatoid arthritis, psoriasis, type I diabetes, uveitis, Celiac disease, pernicious anemia, Srojen's syndrome, Hashimoto's thyroiditis, Graves' disease, systemic lupus erythamatosis, acute disseminated encephalomyelitis, Addison's disease, Ankylosing spondylitis, Antiphospholipid antibody syndrome, Guillain-Barre syndrome, idiopathic thrombocytopenic purpura, Goodpasture's syndrome, Myasthenia gravis, Pemphigus, giant cell arteritis, aplastic anemia, autoimmune hepatitis, Kawaski's disease, mixed connective tissue disease, Ord' throiditis, polyarthritis, primary biliary sclerosis, Reiter's syndrome, Takaysu's arteritis, vitiligo, warm autoimmune hemolytic anemia, Wegener's granulomatosis, Chagas' disease, chronic obstructive pulmonary disease, and sarcoidosis.

Preventing a disease means reducing the likelihood that the disease manifests itself and/or delaying the onset of the disease. Treating a disease means reducing or eliminating the symptoms of the disease.

Examples of orthopedic and other hematological/immunological diseases that may be treated according to the invention are shown in Table 2.

TABLE 2
List of Diseases

Achondroplasia - Acquired Hyperostosis Syndrome - Acrocephalosyndactylia -
Acromegaly - Arthritis - Arthritis, Juvenile Rheumatoid - Arthritis, Rheumatoid - Arthritis,
Reactive - Arthrogryposis - Arthropathy, Neurogenic - Basal Cell Nevus Syndrome -
Bechterew Disease (Spondylitis, Ankylosing) - Bone Diseases - Bone Diseases,
Metabolic - Bone Neoplasms - Bursitis - Cartilage Diseases - Cherubism - Chondromalacia
Patellae - Cleidocranial Dysplasia - Clubfoot - Compartment Syndromes - Congenital
Hypothyroidism - Craniofacial Dysostosis - Craniosynostoses - Dentigerous Cyst -
Dermatomyositis - Dupuytren's Contracture - Dwarfism - Ellis-Van Creveld Syndrome -
Enchondromatosis - Eosinophilia-Myalgia Syndrome -Exostoses - Fasciitis - Fasciitis,
Necrotizing - Fatigue Syndrome, Chronic - Fibromyalgia - Fibrous Dysplasia of Bone -
Fibrous Dysplasia, Polyostotic - Flatfoot - Foot Deformities - Freiberg's Disease (not on
MeSH) - Funnel Chest - Gastroschisis -Gigantism - Goldenhar Syndrome - Gout - Hallux
Valgus - Hip Dislocation, Congenital - Holoprosencephaly - Hyperostosis - Hyperostosis,
Cortical, Congenital - Intervertebral Disk Displacement - Jaw Diseases - Joint Diseases -
Kabuki Make-Up Syndrome (not on MeSH) - Kearns-Sayer Syndrome - Klippel-Feil
Syndrome - Langer-Giedion Syndrome - Legg-Perthes Disease - Lordosis - Mandibulofacial
Dysostosis - Marfan Syndrome - MELAS Syndrome - Melorheostosis - Microcephaly -
Mitochondrial Myopathies - Mucolipidoses - Muscle Cramp - Muscle Spasticity - Muscular
Diseases - Muscular Dystrophies - Musculoskeletal Abnormalities - Musculoskeletal
Diseases - Myopathies, Structural, Congenital - Myositis - Myositis Ossificans - Nail-
Patella Syndrome - Noonan Syndrome - Osteitis Deformans - Osteoarthritis -
Osteochondritis - Osteogenesis Imperfecta - Osteomalacia - Osteomyelitis - Osteonecrosis -
Osteopetrosis - Osteoporosis - Paralyses, Familial Periodic -Pierre Robin Syndrome -
Plagiocephaly, Nonsynostotic - Poland Syndrome - Polychondritis, Relapsing -
Polymyalgia Rheumatica - Polymyositis - Postpoliomyelitis Syndrome - Prognathism -
Proteus Syndrome - Renal Osteodystrophy - Rhabdomyolysis- Rheumatic Diseases -
Rheumatic Fever - Rickets - Rubinstein-Taybi Syndrome - Russell Silver Syndrome (not
on MeSH) - Scoliosis - Scheuermann Disease - Sever's Disease/Calceneal Apophysitis (not
on MeSH) - Sjogren's Syndrome - Spinal Diseases - Spinal Stenosis - Spondylitis,
Ankylosing - Spondylolisthesis - Sprengel's Deformity - Synovitis - Temporomandibular
Joint Disorders - Temporomandibular Joint Dysfunction Syndrome - Tendinopathy - Tennis
Elbow -Tenosynovitis - Thanatophoric Dysplasia - Tietze's Syndrome - Tuberculosis,
Spinal -Multiple sclerosis, type 1 diabetes, rheumatoid arthritis, uveitis, autoimmune
thyroid disease, scleroderma, autoimmune lymphoproliferative disease (ALPS),
demyelinating disease, autoimmune encephalomyelitis, autoimmune gastritis (AIG),
autoimmune glomerular disease, inflammatory bowel disease including Crohn's Disease
and ulcerative colitis, psoriasis, uveitis, Celiac disease, pernicious anemia, Srojen's
syndrome, Hashimoto's thyroiditis, Graves' disease, systemic lupus erythamatosis, acute
disseminated encephalomyelitis, Addison's disease, Ankylosing spondylitis,
Antiphospholipid antibody syndrome, Guillain-Barre syndrome, idiopathic
thrombocytopenic purpura, Goodpasture's syndrome, Myasthenia gravis, Pemphigus, giant
cell arteritis, aplastic anemia, autoimmune hepatitis, Kawaski's disease, mixed connective
tissue disease, Ord' throiditis, polyarthritis, primary biliary sclerosis, Reiter's syndrome,
Takaysu's arteritis, vitiligo, warm autoimmune hemolytic anemia, Wegener's
granulomatosis, Chagas' disease, chronic obstructive pulmonary disease, sarcoidosis, acute
respiratory distress syndrome, systemic inflammatory response syndrome, multiple organ
dysfunction syndrome, sepsis, acute pancreatitis, acute liver failure, acute-on-chronic liver
failure, chronic liver failure, acute renal failure, end stage renal disease, chronic renal
failure, nephrotic syndrome, nephritic syndrome, focal segmental glomerulosclerosis,
glomerulonephritis, acute tubular necrosis, lupus nephritis, diabetic nephritis, interstitial
nephritis, acute-on-chronic renal failure, aplastic anemia, fanconi anemia, hemolytic
anemia, iron-deficient anemia, pernicious anemia, sickle cell anemia, hemochromatosis,
hemophilia, idiopathic thrombocytopenic purpura, polycythemia vera, rh incompatibility,
thalassemias, thrombocytopenia, thrombocythemia, thrombocytosis, thrombophlebitis,
thrombotic thrombocytopenic purpura, Von Willebrand disease, leukemia, lymphatic
filariasis, Anemia From Excessive Bleeding, Anemia of Chronic Disease, Autoimmune
Hemolytic Anemia, Hemoglobin C, S-C, and E Diseases, Vitamin Deficiency Anemia,
Disseminated Intravascular Coagulation (DIC), Henoch-Schonlein Purpura, Hereditary
Hemorrhagic Telangiectasia, Thrombophilia, Acute Lymphocytic Leukemia (ALL), Acute
Myelocytic Leukemia (AML), Chronic Lymphocytic Leukemia (CLL), Chronic Myelocytic
Leukemia (CML), Hodgkin's Disease, Non-Hodgkin's Lymphomas, Myelofibrosis,
Macroglobulinemia, Monoclonal Gammopathies of Undetermined Significance (MGUS),
Multiple Myeloma, Lymphocytopenia, Neutropenia, Neutrophilic Leukocytosis, Chronic
Pain, Migraine, Multiple Abortions

A subject at risk of developing a disease includes one who is genetically predisposed to the disease. Such a subject may have one or more family members that are afflicted with the disease.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops), provided that it would benefit from the methods provided herein. Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. In all embodiments human subjects are preferred. Human subjects can be subjects at any age, including adults, juveniles, infants and fetuses in utero.

The progenitor cells of the invention (and their daughter cells) may be harvested from any subject, whether human or non-human, and may be used in any subject whether human or non-human. Accordingly, the cells may be used in an autologous manner or in a allogeneic manner. In still other embodiments, the cells may be used in a xenogeneic manner.

The invention therefore provides compositions comprising the isolated hematopoietic stromal progenitor cells as specified by the invention. Such compositions may or may not be cryopreserved. Such compositions may be pharmaceutically acceptable such that they are suitable for administration to a subject for diagnostic, prophylactic or therapeutic purpose. The isolated progenitors may be administered to subjects in amounts (or numbers) that are considered to be therapeutically or prophylactically effective, as described herein. The numbers of cells necessary for treatment or prevention will depend on a number of factors including the severity of the symptoms experienced by the subject, the degree of enrichment of the progenitor cells in the administered population, the age and/or weight of the patient, and the like. These factors are well known to those of ordinary skill in the art and can be addressed with no more than routine experimentation.

When administered, the compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The compositions may be administered systemically and/or locally. Local administration includes administration to a site of an aberrant immune response or to a tissue or organ that is affected by a particular condition.

The cells are suspended in and administered with a pharmaceutically acceptable carrier, and in this form are considered pharmaceutical compositions or preparations. As used herein, a pharmaceutically-acceptable carrier means one or more compatible liquid fillers, diluents, and the like, which are suitable for administration into a human. As used herein, carrier means an organic or inorganic ingredient, natural or synthetic, with which the active ingredient is combined to facilitate efficacy and/or administration. The pharmaceutical preparations may contain suitable buffering agents, including but not limited to acetic acid in a salt; citric acid in a salt; boric acid in a salt; and phosphoric acid in a salt. The pharmaceutical compositions also may contain, optionally, suitable preservatives, such as benzalkonium chloride, chlorobutanol, parabens and thimerosal. The components of the pharmaceutical compositions are commingled with cell population, and with each other, in a manner such that there is no interaction which would substantially impair the desired pharmaceutical efficacy. The pharmaceutical compositions or preparations may also comprise other substances including non-cellular agents that either are themselves therapeutically effective or which enhance the therapeutic efficacy of the administered cells. These components may be provided together in a vial, in separate vials in a kit, or in separate kits.

Compositions suitable for administration may comprise a sterile aqueous suspension of cells, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EXAMPLES

Isolation of HSPCs and Cell Culture. Fresh human bone marrow or CD34+ cells were purchased from Lonza Biologics. Mononuclear cells were isolated by Ficoll-Paque™ density separation (GE Healthcare). Bone marrow mononuclear cells were either directly plated or sorted for CD45+ or CD34+ cells using magnetic activated cell sorting (MACS) per vendor's instructions (Milltenyi Biotec). Cells were plated at 10,000 cells/cm² onto tissue culture flask in HSPC expansion medium. HSPC expansion medium consisted of α-MEM (Sigma), 20 mg/L gentamycin (Sigma), 10% FBS (Hyclone), 1 ng/mL rhFGF-basic (R&D Systems), 100 U/ml penicillin (Sigma), and 100 ug/ml streptomycin (Sigma). Non-adherent cells were aspirated on day 7 and the adherent population was cultured for another 4-10 days prior to initial passage. Cells were passaged using 0.1% trypsin/0.1% EDTA, and subcultured at a density of 5×10³ cells/cm². All cultures were used between passages 0-3.

Colony Forming Unit Assays. Limited dilutions of marrow cells were allowed to adhere and proliferate under HSPC expansion for ten days without medium change. The number of adherent colonies was enumerated using a Giemsa stain and visually counted.

In Vitro Differentiation. The multipotency of HSPCs was assessed in vitro by culturing the cells in specified differentiation media as described previously (Lee, Kuo et al. 2004) for 3 weeks with medium changes every 3 days. Cells were stained with Alizarin Red or Oil Red O to assess osteogenic or adipogenic differentiation, respectively.

Flow Cytometry. Cells were stained with BD Pharmigen™ CD44, CD45, CD29, CD73, CD106 or CD11b antibodies (BD Biosciences) after which flow cytometry was performed (Cell Lab Quanta™ SC, Beckman Coulter).

Subcutaneous Transplantation and Ectopic Marrow Formation. This experimental procedure was approved by Subcommittee on Research Animal Care at Massachusetts General Hospital. Adherent cells were suspended in PBS and mixed 1:2 v/v with hydroxyapatite (HA) and tricalcium phosphate (TCP) powders (65:35, HA:TCP w/w; Sigma Aldrich) to establish HA/TCP slurries for each cell population. 150 uL of the slurry was implanted subcutaneously in four sites using an 18 G needle in female nih/nu/xid/bg mice (Harlan). Eight weeks following transplantation, the implants were harvested and prepared for conventional hematoxylin & eosin (H&E) histology. Images were captured on a Nikon Eclipse E800 Upright Microscope.

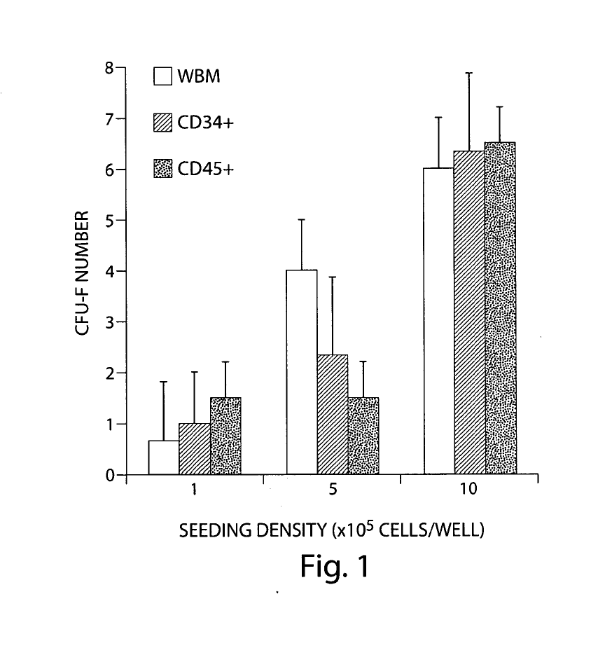

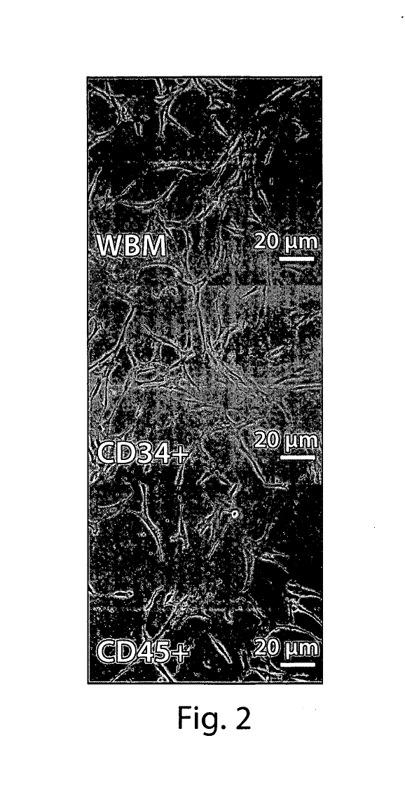

Results and Discussion. Stromal progenitor cells are typically isolated from whole bone marrow mononuclear cells based on differential adhesion. Upon isolation, these cells can be qualitatively described as fibroblastic, nonphagocytic, and able to give rise to colony forming units-fibroblastic (CFU-F) in a clonogenic manner (Bianco and Gehron Robey 2000). To initially evaluate whether or not hematopoietic marrow cells give rise to stromal progenitor cells, we enumerated CFU-F number from whole bone marrow compared to CD34⁺ or CD45⁺ sorted marrow cells. The results of limited dilution assay are shown in FIG. 1. Morphologically, the cells derived from CFU-F displayed a similar fibroblastic morphology consistent with stromal progenitor cells (FIG. 2). Fibrocytes are circulating bone marrow-derived cells that are phenotypically a mixture of monocytes and fibroblasts expressing type I collagen and surface markers that include CD45, CD34, and CD11b (Abe, Donnelly et al. 2001). To ensure these results were not a result of fibrocyte contaminants or other impurities of our sorting methods, we measured CFU-F outgrowths from marrow cells that were purified for CD11b. No CFU-F were observed after plating CD11b+ cells in culture, which validated that the isolated cells were distinct from fibrocytes and our sorting method had insignificant impurities. In addition, we also isolated CFU-F from CD34+ bone marrow cells that were provided by a commercial vendor, which assured ≧95% purity based on their manufacturing processes (data not shown). Taken together, these data suggested that CD34⁺ and CD45⁺ fractions contain clonogenic, fibroblastoid cells.

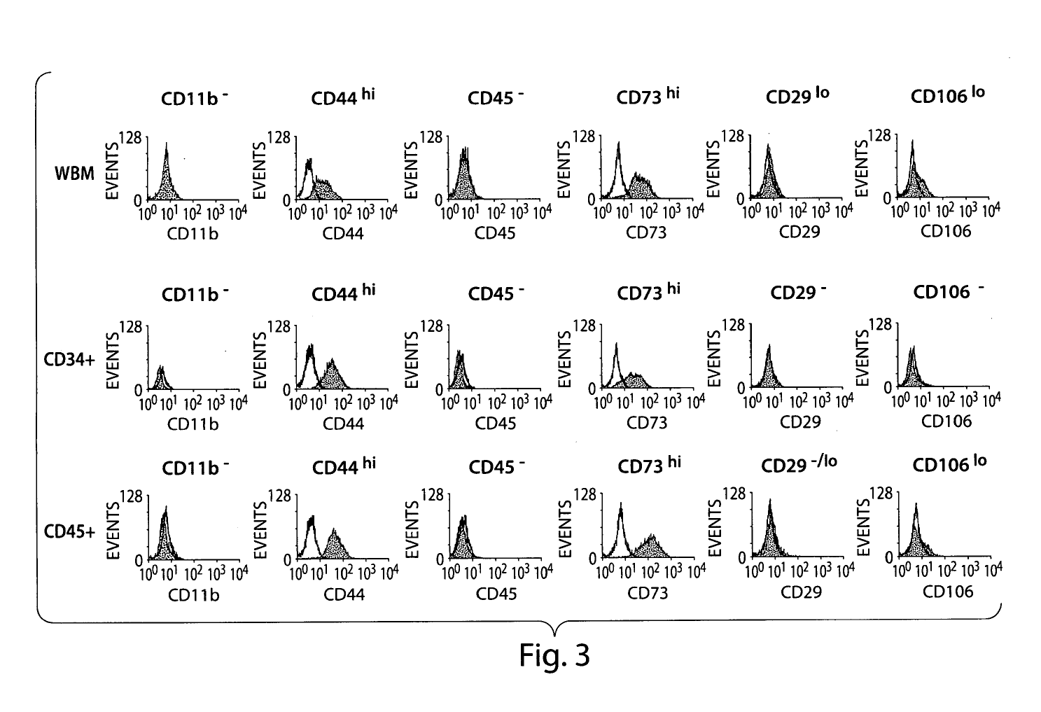

We next evaluated the immunophenotype of adherent cells that were culture expanded from whole bone marrow or sorted marrow populations. The expression of a classical panel of stromal progenitor cell surface markers verified that the isolated fibroblastoid cells were CD44+, CD45−, CD11b−, and CD73+ (FIG. 3). However, unlike stromal progenitor cells, the fibroblastoid cells isolated lost any expression CD29 and CD106 expression, indicative of a phenotypic difference from bona fide MSCs.

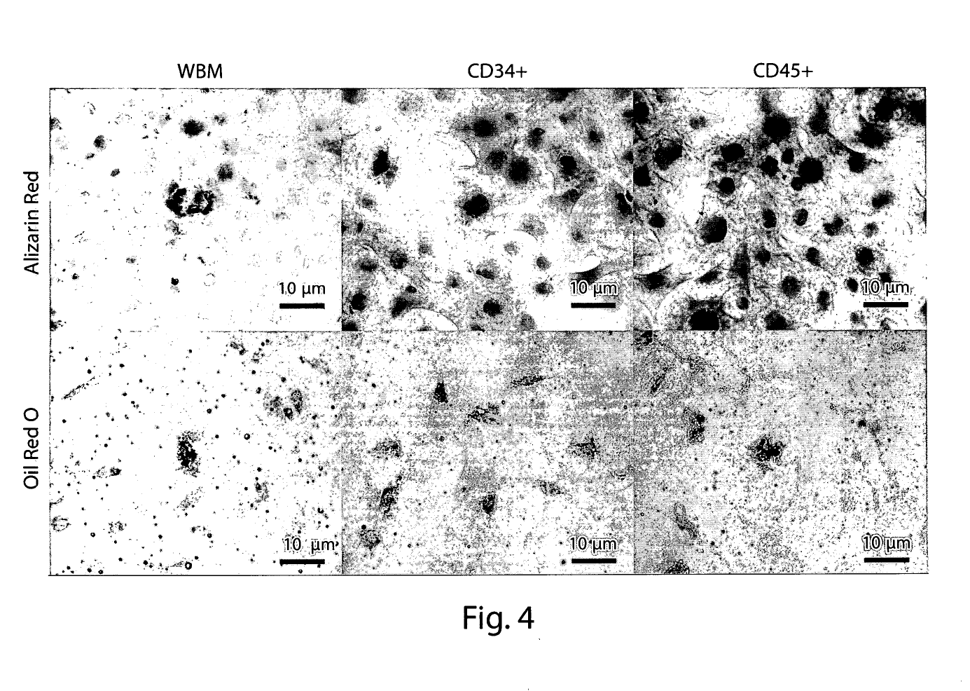

Under certain mechanochemical stimuli, it is reported that CFU-F can give rise to connective tissue cells such as osteoblasts and adipocytes (Friedenstein, Deriglasova et al. 1974; Owen 1988). We measured the capacity for the CFU-F cells derived from CD34+ and CD45+ fractions of bone marrow and whole bone marrow to differentiate into osteogenic and adipogenic lineages by using lineage specifying differentiation medium. As seen in FIG. 4, all three fractions stain positive for differentiation into osseous or adipose cells, respectively. Unexpectedly, the fibroblastoid CFU-F cells exhibited superior differentiation capacity to fat and bone when induced compared to stromal progenitor cells from whole bone marrow, as represented by a higher fraction of differentiated cells.

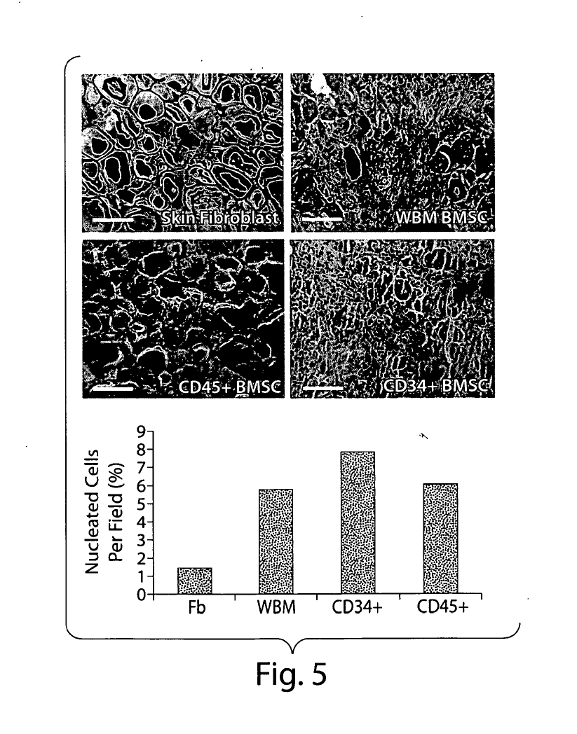

In vivo transplantation of expanded stromal progenitor cells in subcutaneous spaces has been reported to result in the ectopic formation of hematopoietic tissue (Friedenstein, Deriglasova et al. 1974; Sacchetti, Funari et al. 2007). We suspended human stromal progenitor cells derived from whole bone marrow, and the fibroblastoid CFU-F cells derived from the CD34+ or CD45+ cells from bone marrow in hydroxyapatite/tricalcium phosphate slurries and subcutaneously implanted them into immunocompromised mice. Eight weeks after implantation, ectopic bone marrow-like tissue formed using all three preparations, with superior recruitment of hematopoietic tissues in the transplants containing the CFU-F cells (FIG. 5). Similar tissue formation and structure was observed in the whole bone marrow, CD34+, and CD45+, but could not be reproduced using human skin fibroblasts as a mock cell control.

It is clear from these experiments that a distinct cell exists that exhibits similar phenotypic characteristics to stromal progenitor cells, and yet is derived from hematopoietic tissue and expresses a hematopoietic marker, suggesting it is of hematopoietic origin, and is superior in terms of differentiation capacity in vitro and ectopic recruitment of hematopoietic cells in vivo. By isolating these previously unidentified progenitor cells, we have shown that the resultant cultured cells exhibit properties that are significantly different than those found in heterogeneous populations of stromal progenitor cells.

### REFERENCES

Abe, R., S. C. Donnelly, et al. (2001). “Peripheral blood fibrocytes: differentiation pathway and migration to wound sites.” J Immunol 166 (12): 7556-7562.

Aggarwal, S. and M. F. Pittenger (2005). “Human mesenchymal stem cells modulate allogeneic immune cell responses.” Blood 105 (4): 1815-1822.

Au, P., J. Tam, et al. (2008). “Bone marrow-derived mesenchymal stem cells facilitate engineering of long-lasting functional vasculature.” Blood 111 (9): 4551-4558.

Beyth, S., Z. Borovsky, et al. (2005). “Human mesenchymal stem cells alter antigen-presenting cell maturation and induce T-cell unresponsiveness.” Blood 105 (5): 2214-2219.

Bianco, P. and P. Gehron Robey (2000). “Marrow stromal stem cells.” J Clin Invest 105 (12): 1663-1668.

Dierks, C., J. Grbic, et al. (2007). “Essential role of stromally induced hedgehog signaling in B-cell malignancies.” Nat Med 13 (8): 944-951.

Friedenstein, A. J., U. F. Deriglasova, et al. (1974). “Precursors for fibroblasts in different populations of hematopoietic cells as detected by the in vitro colony assay method.” Exp Hematol 2 (2): 83-92.

Horwitz, E. M., D. J. Prockop, et al. (1999). “Transplantability and therapeutic effects of bone marrow-derived mesenchymal cells in children with osteogenesis imperfecta.” Nat Med 5 (3): 309-313.

Hoshida, Y., A. Villanueva, et al. (2008). “Gene expression in fixed tissues and outcome in hepatocellular carcinoma.” N Engl J Med 359 (19): 1995-2004.

Lee, O. K., T. K. Kuo, et al. (2004). “Isolation of multipotent mesenchymal stem cells from umbilical cord blood.” Blood 103 (5): 1669-1675.

Owen, M. (1988). “Marrow stromal stem cells.” J Cell Sci Suppl 10: 63-76.

Parekkadan, B. and J. M. Milwid “Mesenchymal Stem Cells as Therapeutics.” Annu Rev Biomed Eng.

Pittenger, M. F., A. M. Mackay, et al. (1999). “Multilineage potential of adult human mesenchymal stem cells.” Science 284 (5411): 143-147.

Raaijmakers, M. H., S. Mukherjee, et al. “Bone progenitor dysfunction induces myelodysplasia and secondary leukaemia.” Nature 464 (7290): 852-857.

Sacchetti, B., A. Funari, et al. (2007). “Self-renewing osteoprogenitors in bone marrow sinusoids can organize a hematopoietic microenvironment.” Cell 131 (2): 324-336.

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

What is claimed is:

1. An isolated cell population comprising enriched CD34+ human hematopoietic stromal progenitor cells.

2. An isolated cell population comprising enriched CD34+ progenitor cells capable of differentiation into one or more mesenchymal cell lineages.

3. The isolated cell population of claim 1, wherein the isolated cell population is isolated from bone marrow.

4. The isolated cell population of claim 1, wherein the isolated cell population is a human cell population.

5. The isolated cell population of claim 1, wherein the enriched CD34+ human hematopoietic stromal progenitor cells are adherent cells.

6. The isolated cell population of claim 2, wherein the enriched CD34+ progenitor cells capable of differentiation into one or more mesenchymal cell lineages are adherent cells.

7. An in vitro culture comprising the isolated cell population of claim 1.

8. A composition comprising the isolated cell, population of claim 1.

9. A composition comprising the isolated cell population of claim 2.

10. A method comprising
isolating CD34+ cells from a bone marrow cell population,
culturing the isolated CD34+ cells in an in vitro culture, and
separating, from the in vitro culture, adherent cells from non-adherent cells.

11. The method of claim 10, wherein the bone marrow cells are human bone marrow cells.

12. The method of claim 10, further comprising harvesting the adherent cells from the in vitro culture.

13. The method of claim 10, further comprising isolating CD29-negative cells from the adherent cells from the in vitro culture.

14. The method of claim 10, further comprising isolating CD106-negative cells from the adherent cells from the in vitro culture.

15. An isolated cell population prepared by the method of claim 10.

16. A method comprising
administering to a subject in need of a mesenchymal stem transplant a therapeutically effective amount of isolated CD34+ hematopoietic stromal progenitor cells.

17. The method of claim 16, wherein the isolated CD34+ hematopoietic stromal progenitor cells are adherent cells.

18. The method of claim 16, wherein the subject has a connective tissue defect.

19. The method of claim 18, wherein the connective tissue defect is a bone defect.

20. The method of claim 18, wherein the connective tissue defect is a cartilage defect.

21. The method of claim 16, wherein the subject is not in need of a hematopoietic cell transplant.
